(19) United States
(12) Patent Application Publication
(10) Pub. No.: US 2011/0086885 A1
(43) Pub. Date: Apr. 14, 2011

(54) Indole-2-carboxylic acid amides

(76) Inventors: Stuart Edward Bradley, Oxford (GB); Thomas Martin Krulle, Oxford (GB); Gerard Hugh Thomas, Oxfordshire (GB)

(21) Appl. No.: 11/794,939

(22) PCT Filed: Nov. 25, 2005
(86) PCT No.: PCT/GB2005/050214

§ 371 Date: Oct. 1, 2009

(30) Foreign Application Priority Data
Nov. 25, 2004 (GB) 0425919.8

(51) Int. Cl. A61K 31/4439; C07D 209/04; A61K 31/404; C07D 405/12; C07D 401/12; A61P 3/10; A61P 9/12; A61P 9/10

(52) U.S. Cl. 514/339; 548/537; 514/419; 548/526; 514/414; 546/278.1

(57) ABSTRACT

Compounds represented by Formula (I) or pharmaceutically acceptable salts thereof, are useful in the prophylactic or therapeutic treatment of diabetes, hyperglycemia, hypercholesterolemia, hyperinsulinemia, hyperlipidemia, hypertension, atherosclerosis or tissue ischemia e.g. myocardial ischemia, and as cardioprotectants.

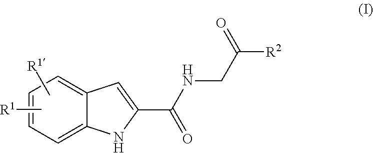

### BACKGROUND OF THE INVENTION

The present invention is directed to indole-2-carboxylic acid amides. In particular, the present invention is directed to, indole-2-carboxylic acid amides that are inhibitors of glycogen phosphorylase.

Insulin dependent Type I diabetes and non-insulin dependent Type II diabetes continue to present treatment difficulties even though clinically accepted regimens that include diet, exercise, hypoglycemic agents, and insulin are available. Treatment is patient dependent—therefore there is a continuing need for novel hypoglycemic agents, particularly ones that may be better tolerated with fewer adverse effects.

The liver and certain other organs produce glucose—thereby raising the blood sugar level—by breaking down glycogen or by synthesizing glucose from small molecule precursors. The breakdown of glycogen is catalyzed by glycogen phosphorylase enzyme. Accordingly, inhibiting glycogen phosphorylase (“GP”) may lower the elevated blood sugar level in diabetic patients.

Similarly, hypertension and its associated pathologies such as, for example, atherosclerosis, lipidemia, hyperlipidemia and hypercholesterolemia have been associated with elevated insulin levels (hyperinsulinemia), which can lead to abnormal blood sugar levels. Furthermore, myocardial ischemia can result. Such maladies may be treated with hypoglycemic agents, including compounds that inhibit glycogen phosphorylase. The cardioprotective effects of glycogen phosphorylase inhibitors, for example following reperfusion injury, has also been described (see, for example, Ross et al., American Journal of Physiology. Heart and Circulatory Physiology, March 2004, 286(3), H1177-84). Accordingly, it is accepted that compounds that inhibit glycogen phosphorylase (see, for example, U.S. Pat. No. 6,297,269) are useful in the treatment of diabetes, hyperglycemia, hypercholesterolemia, hyperinsulinemia, hyperlipidemia, atherosclerosis or myocardial ischemia. Nevertheless, it would be desirable to obtain other novel compounds that inhibit glycogen phosphorylase.

R. Kurukulasuriya, J. T. Link, et al., Current Medicinal Chem., 10:99-121 (2003) describes “Prospects for Pharmacologic Inhibition of Hepatic Glucose Production.” R. Kurukulasuriya, J. T. Link, et al., Current Medicinal Chem., 10:123-153 (2003) describes “Potential Drug Targets and Progress Towards Pharmacologic Inhibition of Hepatic Glucose Production.”

U.S. Pat. No. 6,297,269 and European Patent No. EP 0832066 describe substituted N-(indole-2-carbonyl)amides and derivatives as glycogen phosphorylase inhibitors. U.S. Pat. Nos. 6,107,329 and 6,277,877 describe substituted N-(indole-2-carbonyl)glycinamides and derivatives as glycogen phosphorylase inhibitors. U.S. Pat. No. 6,399,601 describes bicyclic pyrrolyl amides as glycogen phosphorylase inhibitors. European Patent Application Nos. EP 0978276 and EP 1136071 describe inhibitors of human glycogen phosphorylase and their use. International Patent Publication No. WO 01/68055 describes glycogen phosphorylase inhibitors. U.S. Patent Application No. US2004/0002495 describes glycogen phosphorylase inhibitors. U.S. Pat. No. 5,952,322 describes a method of reducing non-cardiac ischemial tissue damage using glycogen phosphorylase inhibitors.

International Patent Publication No. WO 01/55146 describes arylamidines. International Patent Publication No. WO 01/62775 describes antiarrhythmic peptides. International Patent Publication No. WO 01/96346 describes tricyclic compounds. International Patent Publication No. WO 02/16314 describes substituted polyamine compounds. International Patent Publication No. WO 02/20475 describes serine protease activity inhibitors. International Patent Publication No. WO 02/40469 describes bombesin receptor antagonists. International Patent Publication No. WO 02/46159 describes guanidine and amidine derivatives. International Patent Publication No. WO 00/69815 describes ureido-substituted cyclic amine derivatives.

International Patent Publication No. WO 00/43384 describes aromatic heterocyclic compounds. International Patent Publication Nos. WO 02/26697 and WO 00/76970 describe aromatic derivatives. International Patent Publication No. WO 01/32622 describes indoles. European Patent Application No. EP 1101759 describes phenylazole compounds. European Patent Application No. EP 1179341 describes cyclic amino compounds. U.S. Pat. No. 6,037,325 describes substituted heterocyclic compounds. U.S. Pat. No. 5,672,582 describes 4-substituted cyclohexylamine derivatives. European Patent Application No. EP 1201239 describes cyclic amine CCR3 antagonists. International Patent Publication No. WO 98/25617 describes substituted aryl piperazines. U.S. Pat. No. 5,756,810 describes preparing 3-nitrobenzoate compounds.

U.S. Pat. No. 5,710,153 describes tetrazole compounds. U.S. Pat. Nos. 6,174,887 and 6,420,561 describe amide compounds. S. P. Hiremath et al., Acta Ciencia Indica, XVIII: 397 (1992) describes the synthesis and biological activities of indolylthiosemicarbazides and semicarbazides. International Patent Publication No. WO 96/36595 describes 3,4-disubstituted phenylsulfonamides. U.S. Pat. No. 5,618,825 describes combinatorial sulfonamide libraries. European Patent Application No. EP 0810221 describes oxygen-containing heterocyclic derivatives. European Patent Application No. EP 0345990 describes polypeptide compounds. European Patent Application No. EP 0254545 describes diamine compounds.

International Patent Publication No. WO 97/31016 describes inhibitors of SH2-mediated processes. U.S. Pat. No. 6,034,067 describes serine protease inhibitors. International Patent Publication No. WO 97/17985 and U.S. Pat. No. 6,107,309 describe hemoregulatory compounds. U.S. Pat. No. 6,432,921 describes thrombin inhibitors. U.K. Patent Application No. GB 2292149 describes peptide inhibitors of pro-interleukin-1β converting enzyme. U.S. Pat. No. 5,821,241 describes fibrinogen receptor antagonists.

International Patent Publication No. WO 01/02424 describes peptide boronic acid compounds. U.S. Pat. Nos. 6,001,811, 5,869,455 and 5,618,792 describe oxadiazole, thiadiazole and triazole peptoids. U.S. Pat. Nos. 5,885,967, 6,090,787 and 6,124,277 describe thrombin inhibiting peptide derivatives. U.S. Pat. No. 6,455,529 describes adhesion receptor antagonists. U.S. Pat. No. 6,410,684 describes serine protease inhibitors.

International Patent Publication No. WO 01/94310 describes bis-heterocyclic alkaloids. U.S. Patent Publication No. 20030004162A1, European Patent Application No. EP 0846464, and International Publication No. WO 96/39384 describe glycogen phosphorylase inhibitors. International Patent Publication No. WO 97/28798 describes pyrrolidine derivatives. U.S. Pat. No. 5,346,907 describes amino acid analogs. International Patent Application PCT/US2004/016243 (published after the priority date of the present invention) discloses pyrrolopyridine-2-carboxylic acid amide derivatives as glycogen phosphorylase inhibitors.

### SUMMARY OF THE INVENTION

Compounds represented by Formula (I):

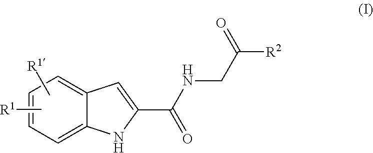

or stereoisomers or pharmaceutically acceptable salts thereof, are inhibitors of glycogen phosphorylase and are useful in the prophylactic or therapeutic treatment of diabetes, hyperglycemia, hypercholesterolemia, hyperinsulinemia, hyperlipidemia, hypertension, atherosclerosis or tissue ischemia e.g. myocardial ischemia, and as cardioprotectants.

### DETAILED DESCRIPTION OF THE INVENTION

The present invention provides a compound of Formula (I):

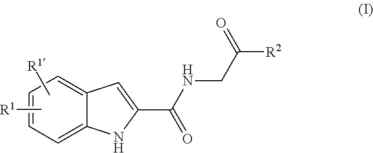

or a stereoisomer, or a pharmaceutically acceptable salt thereof, wherein:
R¹ and R^{1′} are independently selected from hydrogen, halogen, hydroxy, cyano, C₁₋₆alkyl, C₁₋₆alkoxy, fluoromethyl, difluoromethyl, trifluoromethyl, C₂₋₆alkenyl, C₂₋₆alkynyl, aryl, —C₁₋₆alkylaryl, —C₁₋₆alkylheteroaryl or aryloxy;
R² is aryl or heteroaryl, optionally substituted by up to three substituents selected from halogen, hydroxy, cyano, nitro, C₁₋₆alkyl, C₃₋₇cycloalkyl, C₂₋₆alkenyl, C₂₋₆alkynyl, C₁₋₆alkoxy, fluoromethyl, difluoromethyl, trifluoromethyl, methylenedioxo, COOR³ and NR⁴R⁵;
R³ is hydrogen or C₁₋₄alkyl; and
R⁴ and R⁵ are independently selected from hydrogen, C₁₋₄alkyl, aryl and —C₁₋₄alkylaryl;
or R⁴ and R⁵ together with the nitrogen to which they are attached form a 4- to 7-membered heterocycle optionally containing a further heteroatom selected from N and O, which heterocycle is optionally substituted by C₁₋₄alkyl.

The molecular weight of the compounds of Formula (I) is preferably less than 800, more preferably less than 600.

Preferably R¹ and R^{1′} are independently selected from hydrogen, halogen and cyano.

A preferred group of compounds are those where one of R¹ and R^{1′} is hydrogen and the other is a 5-halo or 5-cyano group, especially a 5-chloro group.

R² is preferably phenyl, naphthyl or a 6-membered heteroaryl group e.g. pyridyl such as 3-pyridyl, optionally substituted by up to three substituents selected from halogen e.g. chloro or fluoro, hydroxy, cyano, nitro, C₁₋₆alkyl, C₃₋₇cycloalkyl, C₂₋₆alkenyl, C₂₋₆alkynyl, C₁₋₆alkoxy, fluoromethyl, difluoromethyl, trifluoromethyl, methylenedioxo, COOR³ and NR⁴R⁵.

More preferably R² is phenyl, naphthyl or pyridyl e.g. 3-pyridyl, optionally substituted by one or two substituents, selected from halogen, hydroxy, cyano, nitro, C₁₋₆alkyl, C₃₋₇cycloalkyl, C₂₋₆alkenyl, C₂₋₆alkynyl, C₁₋₆alkoxyl, fluoromethyl, difluoromethyl, trifluoromethyl, methylenedioxo, COOR³ and NR⁴R⁵.

R² is especially phenyl substituted by one or two substituents preferably in the 3- and/or 4-positions, selected from halogen, hydroxy, cyano, nitro, C₁₋₂alkyl, C₁₋₂alkoxy, or methylenedioxo.

Specific compounds of the invention which may be mentioned are those included in the examples, as the free base or a pharmaceutically acceptable salt thereof.

While the preferred groups for each variable have generally been listed above separately for each variable, preferred compounds of this invention include those in which several or each variable in Formula (I) is selected from the preferred, more preferred, especially or particularly listed groups for each variable. Therefore, this invention is intended to include all combinations of preferred, more preferred, most preferred, especially and particularly listed groups.

As used herein, unless stated otherwise, “alkyl” as well as other groups having the prefix “alk” such as, for example, alkoxy, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

As used herein, for example, “C₁₋₄alkyl” is used to mean an alkyl having 1-4 carbons—that is, 0, 1, 2, 3, or 4 carbons in a straight or branched configuration.

The term “cycloalkyl” means carbocycles containing no heteroatoms, and include mono-, bi-, and tricyclic saturated carbocycles, as well as fused and bridged systems. Such fused ring systems can include one ring that is partially or fully unsaturated, such as a benzene ring, to form fused ring systems, such as benzofused carbocycles. Cycloalkyl includes such fused ring systems as spirofused ring systems. Examples of cycloalkyl and carbocyclic rings include C₃₋₇cycloalkyl such as cyclopropyl, cyclobutyl, cyclopentyl and cyclohexyl and the like.

The term “halogen” includes fluorine, chlorine, bromine, and iodine atoms.

The term “aryl” is well known to chemists. The preferred aryl groups are phenyl and naphthyl, more preferably phenyl.

The term “heteroaryl” is well known to chemists. The term includes 5- or 6-membered heteroaryl rings containing 1-4 heteroatoms chosen from oxygen, sulfur, and nitrogen in which oxygen and sulfur are not next to each other. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridyl, pyridazinyl, pyrimidinyl, pyrazinyl, and triazinyl. The term “heteroaryl” includes heteroaryl rings with fused carbocyclic ring systems that are partially or fully unsaturated, such as a benzene ring, to form a benzofused heteroaryl. For example, benzimidazole, benzoxazole, benzothiazole, benzofuran, quinoline, isoquinoline, quinoxaline, and the like.

Unless otherwise stated, the term “heterocycle” includes 4-7-membered saturated or partially saturated rings containing one nitrogen atom and optionally one further heteroatom chosen from oxygen and nitrogen. Any nitrogen heteroatoms in the ring may optionally be substituted with C₁₋₄alkyl. Examples of heterocyclic rings include anticline, oxazolidine, oxazetidine, pyrazolidine, isoxazolidine, azetidine, pyrrolidine, piperidine, N-methylpiperidine, azepane, oxazolidine, piperazine, homopiperazine, morpholine, 1,2,3,6-tetrahydropyridine and the like.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof.

The above Formula (I) is shown without a definitive stereochemistry at certain positions. The present invention includes all stereoisomers of Formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of Formula (I) exists, the present invention includes any possible tautomers and pharmaceutically acceptable salts thereof, and mixtures thereof, except where specifically drawn or stated otherwise.

When the compound of Formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The invention also encompasses a pharmaceutical composition that is comprised of a compound of Formula (I) in combination with a pharmaceutically acceptable carrier.

Preferably the composition is comprised of a pharmaceutically acceptable carrier and a non-toxic therapeutically effective amount of a compound of Formula (I) as described above (or a pharmaceutically acceptable salt thereof).

Moreover, within this preferred embodiment, the invention encompasses a pharmaceutical composition for the treatment of disease by inhibiting glycogen phosphorylase, resulting in the prophylactic or therapeutic treatment of diabetes, hyperglycemia, hypercholesterolemia, hyperinsulinemia, hyperlipidemia, hypertension, atherosclerosis or tissue ischemia e.g. myocardial ischemia comprising a pharmaceutically acceptable carrier and a non-toxic therapeutically effective amount of compound of Formula (I) as described above (or a pharmaceutically acceptable salt thereof).

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salts can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

Since the compounds of Formula (I) are intended for pharmaceutical use they are preferably provided in substantially pure form, for example at least 60% pure, more suitably at least 75% pure especially at least 98% pure (% are on a weight for weight basis).

The pharmaceutical compositions of the present invention comprise a compound represented by Formula (I) (or a pharmaceutically acceptable salt thereof) as an active ingredient, a pharmaceutically acceptable carrier and optionally other therapeutic ingredients or adjuvants. The compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds represented by Formula (I), or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, sachets or tablets each containing a predetermined amount of the active ingredient. Further, the compositions can be presented as a powder, as granules, as a solution, as a suspension in an aqueous liquid, as a non-aqueous liquid, as, an oil-in-water emulsion, or as a water-in-oil liquid emulsion. In addition to the common dosage forms set out above, the compound represented by Formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula (I). The compounds of Formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each sachet or capsule preferably contains from about 0.05 mg to about 5 g of the active ingredient.

For example, a formulation intended for oral administration to humans may contain from about 0.5 mg to about 5 g of active agent, compounded with an appropriate and convenient amount of carrier material, which may vary from about 5 to about 95 percent of the total composition. Unit dosage forms will generally contain from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of Formula (I), or pharmaceutically acceptable salts thereof, may also be prepared in, powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, diabetes and hyperglycemia may be effectively treated by the administration of from about 0.01 to 50 mg of the compound per kilogram of body weight per day, or alternatively about 0.5 mg to about 3.5 g per patient per day. Similarly, hypercholesterolemia, hyperinsulinemia, hyperlipidemia, hypertension, atherosclerosis or tissue ischemia e.g. myocardial ischemia may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of Formula (I) may be used in the treatment of diseases or conditions in which glycogen phosphorylase plays a role.

Thus the invention also provides a method for the treatment of a disease or condition in which glycogen phosphorylase plays a role comprising a step of administering to a subject in need thereof an effective amount of a compound of Formula (I), or a pharmaceutically acceptable salt thereof.

Diseases or conditions in which glycogen phosphorylase plays a role include diabetes (including Type I and Type II, impaired glucose tolerance, insulin resistance and diabetic complications such as neuropathy, nephropathy, retinopathy and cataracts), hyperglycemia, hypercholesterolemia, hyperinsulinemia, hyperlipidemia, hypertension, atherosclerosis, tissue ischemia e.g. myocardial ischemia

The invention also provides a method for the treatment of hyperglycemia or diabetes comprising a step of administering to a subject in need thereof an effective amount of a compound of Formula (I), or a pharmaceutically acceptable salt thereof.

The invention also provides a method for the prevention of diabetes in a human demonstrating pre-diabetic hyperglycemia or impaired glucose tolerance comprising a step of administering to a subject in need thereof an effective prophylactic amount of a compound of Formula (I), or a pharmaceutically acceptable salt thereof.

The invention also provides a method for the treatment of hypercholesterolemia, hyperinsulinemia, hyperlipidemia, hypertension, atherosclerosis or tissue ischemia comprising a step of administering to a patient in need thereof an effective amount of a compound of Formula (I), or a pharmaceutically acceptable salt thereof.

The invention also provides a method of cardioprotection e.g. following reperfusion injury, comprising a step of administering to a subject in need thereof an effective amount of a compound of Formula (I), or a pharmaceutically acceptable salt thereof.

The invention also provides the use of a compound of Formula (I), or a pharmaceutically acceptable salt thereof, in the treatment of a condition as defined above.

The invention also provides the use of a compound of Formula (I), or a pharmaceutically acceptable salt thereof, in the manufacture of a medicament for the treatment of a condition as defined above.

In the methods of the invention the term “treatment” includes both therapeutic and prophylactic treatment.

The compounds of Formula (I), or pharmaceutically acceptable salts thereof, may be administered alone or in combination with one or more other therapeutically active compounds. The other therapeutically active compounds may be for the treatment of the same disease or condition as the compounds of Formula (I) or a different disease or condition. The therapeutically active compounds may be administered simultaneously, sequentially or separately.

The compounds of Formula (I) may be administered with other active compounds for the treatment of diabetes, for example insulin and insulin analogs, sulfonyl ureas and analogs, biguanides, α2 agonists, fatty acid oxidation inhibitors, α-glucosidase inhibitors, β-agonists, phosphodiesterase inhibitors, lipid lowering agents, antiobesity agents, amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, CRF antagonists or CRF binding proteins.

The compounds of Formula (I) may also be administered in combination with thyromimetic compounds, aldose reductase inhibitors, glucocorticoid receptor antagonists, DIRE-1 inhibitors or sorbitol dehydrogenase inhibitors.

The compounds of Formula (I) may exhibit advantageous properties compared to known glycogen phosphorylase inhibitors, for example, the compounds may exhibit improved solubility thus improving absorption properties and bioavailability, or show other advantageous properties which are desirable for pharmaceutical agents.

All publications, including, but not limited to, patents and patent application cited in this specification, are herein incorporated by reference as if each individual publication were specifically and individually indicated to be incorporated by reference herein as fully set forth.

In accordance with this invention, the compounds of Formula (I) can be prepared as outlined in Scheme 1 below wherein R¹, R^{1′} and R² are as defined above for Formula (I):

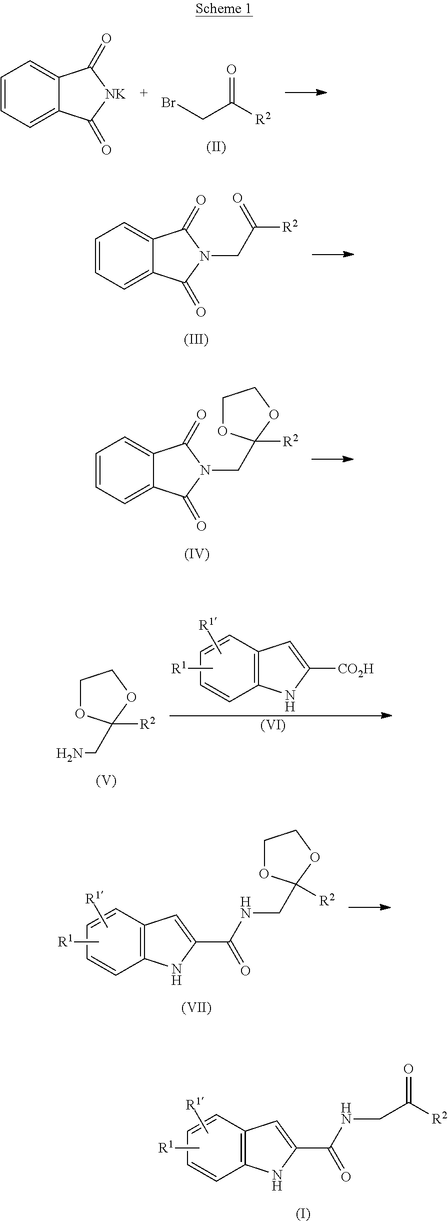

Compounds of Formula (II) are reacted with potassium phthalimide in a solvent such as DMF to give compounds of Formula (III) which is then reacted with ethylene glycol in the presence of a catalytic amount of acid such as p-toluene sulfonic acid in a solvent such as toluene whilst removing water to give compounds of Formula (IV). The phthalimide protecting group is then removed using hydrazine hydrate by heating as a neat solution or by heating in a solvent such as ethanol to give compounds of Formula (V). These amines are then coupled with compounds of Formula (VI), or a protected or activated derivative thereof, under standard amide coupling conditions to give compounds of Formula (VII) and the ketal group removed in the presence of acid, such as hydrochloric acid, in a solvent such as acetone at reflux temperature to give the compounds of Formula (I).

Amide coupling conditions for the coupling of the compounds of Formulae (V) and (VI) include reactions in the presence of a suitable coupling agents. Examples of suitable coupling reagents are 1-(3-dimethylaminopropyl)-3-ethylcarbodiimide hydrochloride/hydroxybenzotriazole (EDCI/HOBt), 1,1-carbonyldiimidazole (CDI), dicyclohexylcarbodiimide/hydroxybenzotriazole (DCC/HOBt), O-(1H-benzotriazol-1-yl)-N,N,N′,N′-tetramethyluronium tetrafluoroborate (R. Knorr et al., Tetrahedron Lett., 1989, 30, 1927-1930) and polymer supported carbodiimide-1-hydroxybenzotriazole (for representative procedures, see for example, Argonaut Technical Note 501 available from Argonaut Technologies, Inc., Foster City, Calif.). The couplings are performed in an inert solvent, preferably an aprotic solvent at a temperature of about 0° C. to about 45° C. for about 1 to 72 h in the presence of a tertiary amine base such as diisopropylethylamine (DIPEA) or triethylamine. Exemplary solvents include acetonitrile, chloroform, dichloromethane, N,N-dimethylformamide (DMF) or mixtures thereof. Use of these coupling agents and appropriate selection of solvents and temperatures are known to those skilled in the art or can be readily determined from the literature. These and other exemplary conditions useful for coupling carboxylic acids are described in Houben-Weyl, Vol XV, part II, E. Wunsch, Ed., G. Thieme Verlag, 1974, Stuttgart, and M. Bodansky, Principles of Peptide Synthesis, Springer-Verlag, Berlin, 1984 and The Peptides, Analysis, Synthesis and Biology (Ed., E. Gross and J. Meienhofer), Vols 1-5, Academic Press NY 1979-1983.

Alternatively the compounds of Formula (I) can be prepared by oxidation of a compound of Formula (VIII) wherein R¹, R^{1′} and R² are as defined above for Formula (I):

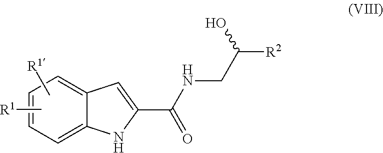

Suitable oxidation conditions include oxidation with Dess-Martin periodinane.

Compounds of Formula (VIII) may be prepared by coupling of amines of Formula (IX) with compounds of Formula (VI), or a protected or activated derivative thereof, under standard amide coupling conditions as described above:

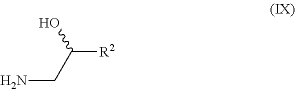

The compounds of Formulae (II), (VI) and (IX) are known or may be prepared by methods known to those skilled in the art. Protected or activated derivatives of the compounds of Formula (VI) may also be prepared by methods known to those skilled in the art.

The compounds of Formula (I) may be prepared singly or as compound libraries comprising at least 2, for example 5 to 1,000 compounds and more preferably 10 to 100 compounds of Formula (I). Compound libraries may be prepared by a combinatorial “split and mix” approach or by multiple parallel synthesis using either solution or solid phase chemistry, using procedures known to those skilled in the art.

During the synthesis of the compounds of Formula (I), labile functional groups in the intermediate compounds, e.g. hydroxy, carboxy and amino groups, may be protected. The compounds of Formula (VI) may be protected in the 1-position e.g. with an arylmethyl, acyl, alkoxycarbonyl, sulfonyl or silyl group. The protecting groups may be removed at any stage in the synthesis of the compounds of Formula (I) or may be present on the final compound of Formula (I). A comprehensive discussion of the ways in which various labile functional groups may be protected and methods for cleaving the resulting protected derivatives is given in for example, Protective Groups in Organic Chemistry, T. W. Greene and P. G. M. Wuts, (1991) Wiley-Interscience, New York, 2^{nd} edition.

Any novel intermediates as defined above, e.g. intermediates of Formula (VII) and (VIII), are also included within the scope of the invention.

Thus according to a further aspect of the invention there is provided a compound of Formula (VII), or a protected derivative thereof:

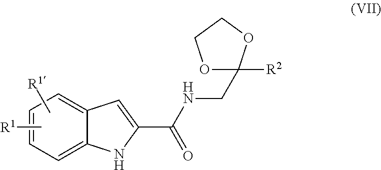

wherein R¹, R^{1′} and R², and the preferences therefore, are as defined above for Formula (I).

Thus according to a further aspect of the invention there is provided a compound of Formula (VIII), or a protected derivative thereof:

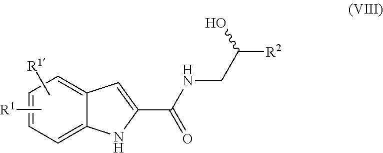

wherein R¹, and R², and the preferences therefore, are as defined above for Formula (I); provided that the compound is not 5-fluoro-1H-indole-2-carboxylic acid [2-hydroxy-2-phenylethyl]amide.

### EXPERIMENTAL

### Materials & Methods

Column chromatography was carried out on SiO₂ (40-63 mesh). LCMS data were obtained using a Waters Symmetry 3.5μ C₁₈ column (2.1×30.0 mm, flow rate=0.8 mL/min) eluting with a (5% MeCN in H₂O)-MeCN solution containing 0.1% HCO₂H over 6 min and UV detection at 220 nm. Gradient information: 0.0-1.2 min: 100% (5% MeCN in H₂O); 1.2-3.8 min: ramp up to 10% (5% MeCN in H₂O)-90% MeCN; 3.8-4.4 min: hold at 10% (5% MeCN in H₂O)-90% MeCN; 4.4-5.5 min: ramp up to 100% MeCN; 5.5-6.0 min: return to 100% (5% MeCN in H₂O). The mass spectra were obtained employing an electrospray ionisation source in the positive (ES⁺) ion mode. NMR spectra were acquired at 27° C. on a Varian Mercury 400 spectrometer operating at 400 MHz or on a Bruker AMX2 500 spectrometer operating at 500 MHz. Mass directed purification was performed on a Micromass Platform LC with cone voltage 30 v, employing an electrospray ionisation source in the positive (ES⁺) ion mode, Waters 996 Photodiode Array Detector (210-390 nm), Xterra Prep MS, C₁₈, 5μ 19×50 mm columns, and a mobile Phase of MeCN+0.1% Formic Acid/H₂0+5% MeCN+0.1% Formic Acid

Abbreviations and acronyms: BOC: tert-Butyloxycarbonyl; DBU: 1,8-Diazabicyclo[5.4.0]undec-7-ene; DCM: Dichloromethane; DIPEA: N,N-Diisopropylethylamine; DMF: N,N-Dimethylformamide; DMSO: Dimethylsulfoxide; DMTMM: 4-(4,6-Dimethoxy[1.3.5]triazin-2-yl)-4-methylmorpholinium chloride hydrate; EDCI: 1-(3-Dimethylaminopropyl)-3-ethylcarbodiimide hydrochloride; GP: Glycogen Phosphorylase; HATU: O-(7-Azabenzotriazole-1-yl)-N,N,N′,N′-tetramethyluronium hexafluorophosphate; HOBt: 1-Hydroxybenzotriazole; MDP: Mass directed purification; MgSO₄: Magnesium sulfate; PS: Polymer supported; rt: Room temperature; RT: Retention time; THY: Tetrahydrofuran, TBTU: O-(Benzotriazol-1-yl) N,N,N′,N′-tetramethyluronium tetrafluoroborate

### Preparation 1: 2-[2-(4-Methoxyphenyl)-2-oxoethyl]isoindole-1,3-dione

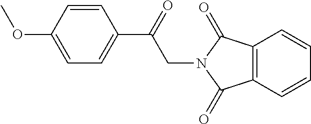

To a solution of 4-methoxyphenacyl bromide (5.78 g, 25.23 mmol) in DMF (20 mL) was added potassium phthalimide (5.00 g, 26.99 mmol) and the reaction stirred at rt for 18 h. The reaction mixture was partitioned between DCM (200 mL) and water (100 mL). The layers were separated and the aqueous layer extracted with DCM (3×50 mL). The combined organics were washed with sodium hydroxide (2M, 50 mL), water (50 mL) and brine (50 mL) and dried (MgSO₄). Filtration, then concentration in vacuo gave an off white solid. Trituration with diethyl ether followed by collection by filtration gave the title compound. δ_{H} (CDCl₃): 3.86 (3H, s), 5.15 (2H, s), 7.09 (2H, d), 7.81-8.01 (4H, m), 8.05 (2H, d).

The following compounds were synthesised according to Preparation 1 from potassium phthalimide and the appropriate α-bromoketone:

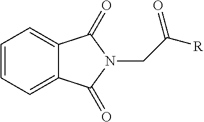

| Preparation | R | NMR |
|---|---|---|
| 2 | 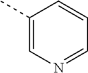 | δ_{H} (d₆-DMSO): 5.32 (2H, s), 7.63 (1H, dd), 7.85-8.02 (4H, m), 8.42 (1H, d), 8.88 (1H, d), 9.27 (1H, s) |
| 3 | 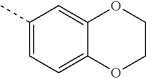 | δ_{H} (d₆-DMSO): 4.24-4.43 (4H, m), 5.13 (2H, s), 7.03 (1H, d), 7.58 (1H, d), 7.61 (1H, d), 7.83-8.02 (4H, m) |
| 4 | 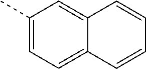 | δ_{H} (d₆-DMSO): 5.38 (2H, s), 7.63-7.76 (2H, m), 7.87-8.10 (7H, m), 8.16 (1H, d), 8.90 (1H, s) |
| 5 | 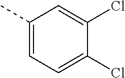 | δ_{H} (d₆-DMSO): 5.28 (2H, s), 7.83-7.99 (5H, m), 8.03 (1H, dd), 8.33 (1H, d) |
| 6 | 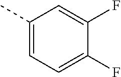 | δ_{H} (d₆-DMSO): 5.25 (2H, d), 7.60-7.75 (1H, 7.85-8.06 (5H, m), 8.10-8.24 (1H, m) |
| 7 | 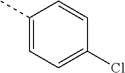 | δ_{H} (d₆-DMSO): 5.24 (2H, s), 7.66 (2H, d), 7.83- 8.02 (4H, m), 8.10 (2H, d) |
| 8 | 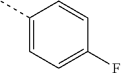 | δ_{H} (d₆-DMSO): 5.24 (2H, s), 7.36-7.52 (2H, m), 7.84-8.02 (4H, m), 8.11-8.25 (2H, m) |

### Preparation 9: 2-[2-(4-Methoxyphenyl)-[1,3]dioxolan-2-ylmethyl]isoindole-1,3-dione

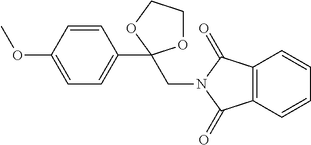

2-[2-(4-Methoxyphenyl)-2-oxoethyl]isoindole-1,3-dione (Preparation 1, 635 g, 21.5 mmol) was suspended in toluene (50 mL) and ethylene glycol (12 mL) added. p-Toluenesulfonic acid (300 mg, 1.58 mmol) was added and the resulting mixture heated under reflux for 40 h, removing water with a Dean-Stark trap. The reaction mixture was allowed to cool to rt then partitioned between ethyl acetate (200 mL) and saturated aqueous sodium bicarbonate solution (100 mL). The organic layer was washed with brine (50 mL), dried (MgSO₄), filtered and concentrated under reduced pressure, to give the title compound. δ_{H} (d₆-DMSO): 3.66 (2H, t), 3.72 (3H, s), 3.87 (4H, m), 6.88 (2H, d), 7.32 (2H, d), 7.83 (4H, m).

The following compounds were synthesised according to Preparation 9 from ethylene glycol and the appropriate ketone:

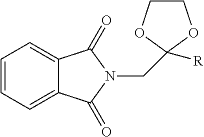

| Preparation | R | NMR |
|---|---|---|
| 10 | 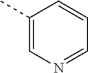 | δ_{H} (d₆-DMSO): 3.73-3.81 (2H, m), 3.96 (2H, s), 3.97-4.05 (2H, m), 7.36 (1H, dd), 7.75-7.79 (1H, m), 7.80-7.86 (4H, m), 8.52 (1H, dd), 8.57 (1H, d) |
| 11 | 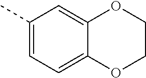 | δ_{H} (d₆-DMSO): 3.66 (2H, t), 3.84 (2H, t), 3.86 (2H, s), 4.21 (4H, s), 6.72-6.94 (3H, m), 7.76-7.95 (4H, m) |
| 12 | 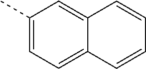 | δ_{H} (d₆-DMSO): 3.72-3.78 (2H, m), 3.94-3.99 (2H, m), 4.01 (2H, s), 7.45-7.63 (3H, m), 7.79-7.87 (4H, m), 7.87-8.02 (4H, m) |
| 13 | 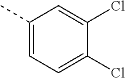 | δ_{H} (d₆-DMSO): 3.71-3.80 (2H, m), 3.93 (2H, s), 3.95-4.00 (2H, m), 7.36 (1H, dd), 7.57 (1H, d), 7.61 (1H, d), 7.79-7.90 (4H, m) |
| 14 | 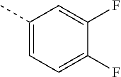 | δ_{H} (d₆-DMSO): 3.69-3.81 (2H, m), 3.85-4.04 (4H, m), 7.15-7.30 (1H, m), 7.32-7.49 (2H, m), 7.74-7.96 (4H, m) |
| 15 | 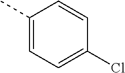 | δ_{H} (d₆-DMSO): 3.62-3.78 (2H, m), 3.84-3.99 (4H, m), 7.32-7.49 (4H, m), 7.74-7.94 (4H, m) |
| 16 | 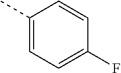 | δ_{H} (d₆-DMSO): 3.64-3.80 (2H, m), 3.85-4.03 (4H, m), 7.08-7.24 (2H, m), 7.35-7.52 (2H, m), 7.75-7.93 (4H, m) |

### Preparation 17: [2-(4-Methoxyphenyl)-[1,3]dioxolan-2-yl]methylamine

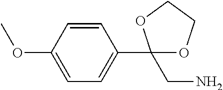

2-[2-(4-Methoxyphenyl)-[1,3]dioxolan-2-ylmethyl]isoindole-1,3-dione (Preparation 9, 2.0 g, 5.90 mmol) and hydrazine hydrate (5 mL) were combined. The stirred reaction mixture was heated under reflux for 48 h then allowed to cool to rt. Aqueous sodium hydroxide (2M, 10-15 mL) and water (20 mL) were added and the mixture stirred until a solution was formed. Diethyl ether (20 mL) was added and the biphasic mixture stirred vigorously for 16 h. The layers were separated and the aqueous layer was extracted with diethyl ether (3×20 mL), then the combined organic extracts were washed with brine (20 mL). The ethereal solution was passed through a filter paper then evaporated to dryness in vacuo to a yellow oil, which solidified on standing, to give the title compound. δ_{H} (CDCl₃): 1.42 (2H, br s), 3.06 (2H, s), 3.97 (3H, s), 4.00 (2H, t), 4.21 (2H, t), 7.04 (2H, d), 7.53 (214, d).

The following compounds were synthesised according to Preparation 17 from hydrazine hydrate and the corresponding phthalimide:

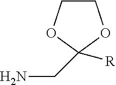

| Preparation | R | NMR |
|---|---|---|
| 18 | 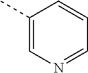 | δ_{H} (CDCl₃): 1.58 (2H, br s), 2.87 (2H, s), 3.75-3.83 (2H, m), 4.00-4.07 (2H, m), 720-7.24 (1H, m), 7.70 (1H, dt), 8.51 (1H, dd), 8.65 (1H, d) |
| 19 | 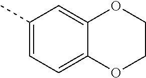 | δ_{H} (CDCl₃): 1.25 (2H, br s), 2.87 (2H, s), 3.78-3.88 (2H, m), 3.97-4.06 (2H, m), 4.24 (4H, s), 6.81 (1H, d), 6.90 (1H, dd), 6.95 (1H, d) |
| 20 | 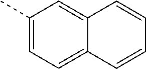 | δ_{H} (CDCl₃): 1.43 (2H, br s), 3.00 (2H, s), 3.81-3.91 (2H, m), 4.02-4.11 (2H, m), 7.43-7.50 (2H, m), 7.54 (1H, d), 7.76-7.87 (3H, m), 7.94 (1H, s) |
| 21 | 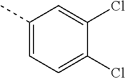 | δ_{H} (CDCl₃): 1.26 (2H, br s), 2.87 (2H, s), 3.77-3.88 (2H, m), 4.01-4.11 (2H, m), 7.27 (1H, dd), 7.41 (1H, d), 7.54 (1H, d) |
| 22 | 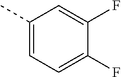 | δ_{H} (CDCl₃): 1.31 (2H, br s), 2.87 (2H, s), 3.79-3.87 (2H, m), 3.99-4.08 (2H, m), 7.00-7.20 (2H, m), 7.23-7.29 (1H, m) |
| 23 | 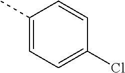 | δ_{H} (CDCl₃): 1.23 (2H, br s), 2.89 (2H, s), 3.75-3.87 (2H, m), 3.99-4.10 (2H, m), 729-7.33 (2H, m), 7.36-7.40 (2H, m) |
| 24 | 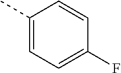 | δ_{H} (d₆-DMSO): 2.72 (2H, s), 3.25 (2H, br s), 3.67-3.78 (2H, m), 3.93-4.06 (2H, m), 7.11-7.18 (2H, m), 7.37-7.44 (2H, m) |

### Preparation 25: 5-Chloro-1H-indole-2-carboxylic acid [2-(4-methoxyphenyl)-[1,3]dioxolan-2-ylmethyl]amide

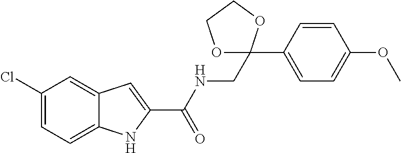

To a solution of [2-(4-methoxyphenyl)-[1,3]dioxolan-2-yl]methylamine in dichloromethane (Preparation 17, 4.5 mL, anhydrous) was added DIPEA (213 μL, 1.22 mmol) then 5-chloroindole-2-carboxylic acid (100 mg, 0.51 mmol). HOBt.H₂O (76 mg, 0.56 mmol) was added and the reaction mixture stirred for 2-3 min prior to the addition of WSC (117 mg, 0.61 mmol). Stirring was continued for 18 h at rt then the reaction mixture partitioned between DCM (20 mL) and water (20 mL). The layers were separated and the aqueous layer extracted with DCM (3×20 mL). The combined organics were washed with brine (20 mL), dried (MgSO₄), filtered and concentrated in vacuo. Recrystallisation was achieved using DCM/methanol and the resulting solid was filtered and washed with diethyl ether to afford the title compound. RT=3.77 min.

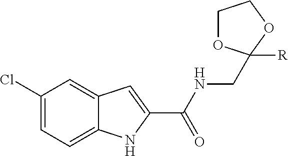

The following compounds were prepared according to Preparation 25 from 5-chloroindole-2-carboxylic acid and the appropriate α-aminoketal:

| Preparation | R | RT |
|---|---|---|
| 26 | 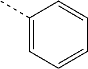 | RT = 3.75 min |
| 27 | 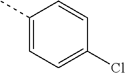 | RT = 1.81 min |
| 28 | 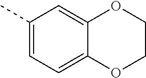 | RT = 3.62 min |
| 29 | 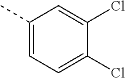 | RT = 1.88 min |
| 30 | 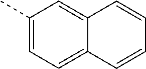 | RT = 1.83 min |
| 31 | 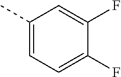 | RT = 1.75 min |

### Preparation 32: 5-Chloro-1H-indole-2-carboxylic acid [2-(4-fluorophenyl)-[1,3]dioxolan-2-ylmethyl]amide

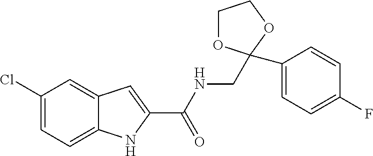

To a solution of [2-(4-fluorophenyl)-[1,3]dioxolan-2-yl]methylamine (Preparation 24, 200 mg, 1.02 mmol) in DMF (5 mL, anhydrous) was added 5-chloroindole-2-carboxylic acid (180 mg, 0.92 mmol), DIPEA (407 μL, 2.33 mmol) then HOBt.H₂O (137 mg, 1.02 mmol). The reaction mixture was stirred for 10 min prior to the addition of WSC (212 mg, 1.11 mmol) then for 64 h at room temperature. The volatiles were removed under reduced pressure then the residue partitioned between DCM (30 mL) and water (20 mL) then passed through a hydrophobic frit. The aqueous phase was washed twice with DCM (2×20 mL) then the combined organics were evaporated to dryness and the resulting residue triturated with ethyl acetate. The suspension was filtered and the solid washed with ethyl acetate (10 mL) then air dried to give the desired compound. RT=3.72 min.

### Preparation 33: 2-Amino-1-pyridin-3-ylethanol

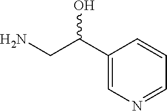

To a solution of crude 2-(2-oxo-2-pyridin-3-ylethyl)isoindole-1,3-dione (Preparation 2, 5.0 g, ˜19.0 mmol) in aqueous isopropanol (210 ml, water/IPA: 1/6) was added sodium borohydride (10.2 g, 270 mmol) in 2 portions. The mixture was stirred at rt for 12 h before carefully acidified (pH 2) with dilute hydrochloric acid (1M). After removal of the solvent the residue was taken up in distillated water (100 mL) and passed down a column filled with ion-exchange resin (Amberlite IR 120, H⁺-form, 300 g; eluent: 500 mL water then 1 L of 2 M aqueous ammonia solution). Concentration of the alkaline fractions gives the title compound. δ_{H} (d₆-DMSO): 2.74, 2.85 (2H, 2m), 4.66 (1H, m), 5.15 (3H, br s), 7.36 (1H, dd), 7.75 (1H, m), 8.46 (1H, m), 8.56 (1H, m); m/z (ES⁺)=139.11 [M+H]⁺; RT=0.21 min.

### Preparation 34: 5-Chloro-1H-indole-2-carboxylic acid [2-hydroxy-2-pyridin-3-yl-ethyl]amide

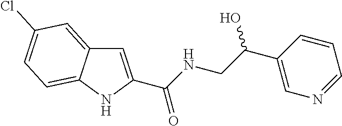

To a solution of 5-chloro-1H-indole-2-carboxylic acid (295 mg, 1.51 mmol) and 2-amino-1-pyridin-3-ylethanol (Preparation 33, 220 mg, 1.59 mmol) in DMF (5 mL) was added HOBt (225 mg, 1.47 mmol), DIPEA (0.56 mL, 3.21 mmol) and EDCI (340 mg, 1.77 mmol). After stirring at rt for 12 h the solvent was removed in vacuo and the residue was purified by flash chromatography on silica gel (eluent: DCM/methanol: 90/10). The fractions containing product and/or the hydrochloride of the product were concentrated before taken up in DCM (150 mL). Washing with dilute NaOH solution (1 M, 50 ml) and brine (50 mL), drying (MgSO₄) and concentration in vacuo afforded the title compound as off-white solid. δ_{H} (d₆-DMSO): 3.52 (2H, m), 4.83 (1H, dt), 5.72 (1H, d), 7.10 (1H, s), 7.18 (1H, dd), 7.36 (1H, dd), 7.42 (1H, d), 7.69 (1H, s), 7.76 (1H, d), 8.45 (1H, m), 8.55 (1H, s), 8.61 (1H, t), 11.74 (1H, s); m/z (ES⁺)=316.18 [M+H]⁺; RT=2.52 min.

### Preparation 34: 5-Chloro-1H-indole-2-carboxylic acid [2-hydroxy-2-(4-nitro-phenyl)ethyl]amide

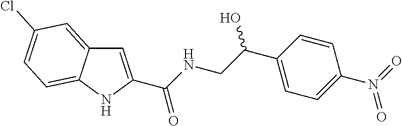

To a solution of 5-chloro-1H-indole-2-carboxylic acid (508 mg, 2.60 mmol) and 2-amino-1-(4-nitrophenyl)ethanol (480 mg, 2.63 mmol) in DMF (10 mL) was added HOBt (400 mg, 2.61 mmol), DIPEA (0.98 mL, 5.63 mmol) and EDCI (580 mg, 3.03 mmol). After stirring at it for 12 h the reaction mixture was partitioned between ethyl acetate (50 mL) and water/brine (150 mL, 1:1). The layers were separated and the aqueous phase extracted with ethyl acetate (3×50 mL), then the combined organics were washed with dilute HCl solution (1M, 50 mL), dilute NaOH solution (1M, 50 mL) and brine (50 mL). The organic phase was dried (MgSO₄), filtered and concentrated to a solid residue, which was washed with a small amount of chloroform to give the title compound. δ_{H} (d₆-DMSO): 3.41-3.58 (2H, m), 4.94 (1H, dt), 5.90 (1H, d), 7.11 (1H, s), 7.17 (1H, dd), 7.41 (1H, d), 7.67 (2H, m), 7.70 (1H, s), 8.21 (2H, m), 8.65 (1H, t), 11.74 (1H, s); m/z (ES⁺)=360.19 [M+H]⁺; RT=3.49 min.

### Example 1

### 5-Chloro-1H-indole-2-carboxylic acid [2-(4-methoxyphenyl)-2-oxoethyl]amide

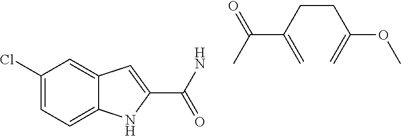

5-Chloro-1H-indole-2-carboxylic acid [2-(4-methoxyphenyl)[1,3]dioxolan-2-ylmethyl]amide (Preparation 26, 130 mg, 0.34 mmol) was suspended in acetone (20 mL) and 2M HCl (1.7 mL, 3.4 mmol) was added. The reaction mixture was heated to reflux temperature for 90 min then cooled to rt and filtered through a sinter. The solid was washed with acetone then air dried to furnish the desired compound. RT=3.68 min; δ_{H} (d₆-DMSO): 3.85 (3H, s), 4.76 (2H, d), 7.07 (2H, d), 7.18 (1H, s), 7.19 (1H, s), 7.43 (11.1, d), 7.72 (1H, s), 8.03 (2H, d), 8.86 (1H, t), 11.80 (1H, s).

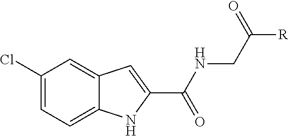

The following compounds were prepared according to Example 1 using the corresponding ketal.

| Example | R | RT / NMR |
|---|---|---|
| 2 | 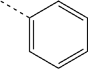 | RT = 3.67 min; δ_{H} (d₆-DMSO): 4.81 (2H, t), 7.11- 7.26 (2H, m), 7.39-7.48 (1H, m), 7.50-7.61 (2H, m), 7.62-7.79 (2H, m), 7.96-8.12 (2H, m), 8.84-9.00 (1H, m), 11.80 (1H, s). |
| 3 | 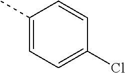 | RT = 3.65 min; δ_{H} (d₆-DMSO): 4.80 (2H, d), 7.19 m), 7.42 (1H, d), 7.63 (2H, d), 7.72 (1H, s), 8.06 (2H, d), 8.94 (1H, t), 11.80 (1H, s). |
| 4 | 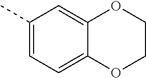 | RT = 3.96min; δ_{H} (d₆-DMSO): 4.27-4.31 (2H, m), 4.32-4.37 (2H, m), 4.72 (2H, d), 7.00 (1H, d), 7.15-7.18 (1H, m), 7.19 (1H, d), 7.43 (1H, d), 7.54 (1H, d), 7.58 (1H, dd), 7.72 (1H, d), 8.87 (1H, t), 11.80 (1H, s). |
| 5 | 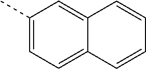 | RT = 4.02 min; δ_{H} (d₆-DMSO): 4.97 (2H, d), 7.19 (1H, d), 7.21 (1H, s), 7.44 (1H, d), 7.62-7.72 (2H, m), 7.73 (1H, s), 7.98-8.09 (3H, m), 8.15 (1H, d), 8.81 (1H, s), 8.98 (1H, t), 11.83 (1H, s). |
| 6 | 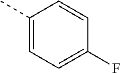 | RT = 3.54 min; δ_{H} (d₆-DMSO): 4.80 (2H, d), 7.18 (1H, s), 7.20 (1H, d), 7.35-7.47 (3H, m), 7.72 (1H, d), 8.13 (2H, dd), 8.93 (1H, t), 11.80 (1H, s). |

### Example 7

### 5-Chloro-1H-indole-2-carboxylic acid [2-(3,4-dichlorophenyl)-2-oxo-ethyl]amide

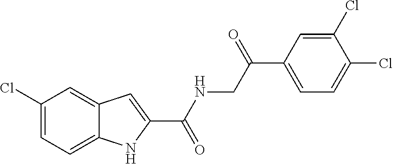

5-Chloro-1H-indole-2-carboxylic acid [2-(3,4-dichlorophenyl)-[1,3]dioxolan-2-ylmethyl]amide (Preparation 29, 120 mg, 0.28 mmol) was suspended in TFA (3 mL), acetonitrile (10 mL) and water (2 mL) and the reaction mixture heated to reflux temperature for 18 h. The reaction mixture was cooled to rt then the volatiles removed in vacuo. To the resulting wet residue was added acetone (10 mL) and the suspension filtered through a sinter, washing with acetone then the resulting solid was air dried to give the desired compound as a beige solid. RT=4.14 min; δ_{H} (d₆-DMSO): 4.89 (2H, d), 7.23-7.31 (2H, m), 7.51 (1H, d), 7.80 (1H, s), 7.92 (1H, d), 8.07 (1H, d), 8.34 (1H, s), 9.05 (1H, t), 11.88 (1H, s).

### Example 8

### 5-Chloro-1H-indole-2-carboxylic acid [2-(3,4-difluorophenyl)-2-oxo-ethyl]amide

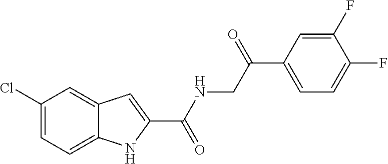

5-Chloro-1H-indole-2-carboxylic acid [2-(3,4-difluorophenyl)-[1,3]dioxolan-2-ylmethyl]amide (Preparation 31, 77 mg, 0.20 mmol) was suspended in TFA (3 mL), acetonitrile (10 mL) and water (2 mL) and the reaction mixture heated to reflux temperature for 18 h. The reaction mixture was cooled to rt then the volatiles removed in vacuo. To the resulting wet residue was added acetone (10 mL) and the suspension filtered through a sinter, washing with acetone then the resulting solid was air dried to give the desired compound as a yellow solid. RT=3.99 min; δ_{H} (d₆-DMSO): 4.8 (2H, d), 7.16-7.21 (2H, m), 7.43 (1H, d), 7.60-7.68 (1H, m), 7.72 (1H, s), 7.92-7.98 (1H, m), 8.07-8.15 (1H, m), 8.95 (1H, t), 11.80 (1H, s).

### Example 9

### 5-Chloro-1H-indole-2-carboxylic acid [2-oxo-2-pyridin-3-ylethyl]amide

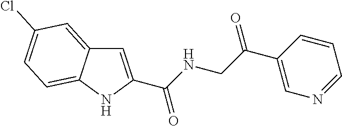

To a suspension of racemic 5-chloro-1H-indole-2-carboxylic acid [2-hydroxy-2-pyridin-3-ylethyl]amide (Preparation 33, 183 mg, 0.58 mmol) in dry DCM (5 mL) was added Dess-Martin periodinane (303 mg, 0.71 mmol). After stirring for 2 h at rt sodium thiosulfate solution was added (5.4 g Na₂S₂O₃ dissolved in 20 mL water) and the emulsion was vigorously stirred for additional 10 min before further diluted with saturated sodium hydrogen carbonate solution (˜150 mL). Extraction with DCM (3×50 mL), washing of the combined extracts with saturated sodium hydrogen carbonate solution (50 mL) and brine (50 mL) gave after drying (MgSO₄) and concentration in vacuo a solid residue, which was recrystallised from THF to give the title compound as colourless crystals. δ_{H} (d₆ DMSO): 4.85 (2H, m), 7.18-7.21 (2H, m), 7.44 (1H, d), 7.61 (1H, dd), 7.73 (1H, s), 8.38 (1H, d), 8.84 (1H, m), 9.00 (1H, t), 9.22 (1H, s), 11.81 (1H, s); (ES⁺)=314.16 [M+H]⁺; RT=3.21 min.

### Example 10

### 5-Chloro-1H-indole-2-carboxylic acid [2-(4-nitrophenyl)-2-oxo-ethyl]amide

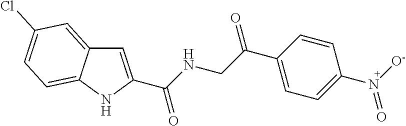

To a solution of racemic 5-chloro-1H-indole-2-carboxylic acid [2-hydroxy-2-(4-nitrophenyl)ethyl]amide (Preparation 34, 104 mg, 0.289 mmol) in dry DCM (5 mL) was added Dess-Martin periodinane (148 mg, 0.349 mmol). After stirring for 12 h at rt alkaline sodium thiosulfate solution was added (2.7 g Na₂S₂O₃ dissolved in 10 mL saturated NaHCO₃ solution) and the emulsion was vigorously stirred for additional 5 min before further diluted with water (˜100 mL). Extraction with ethyl acetate (4×50 mL), washing of the combined extracts with saturated sodium hydrogen carbonate (50 mL) and brine (50 mL) gave after drying (MgSO₄) and concentration in vacuo a solid residue, which was washed with a small amount of chloroform to give the title compound. δ_{H} (d₆ DMSO): 4.94 (2H, m), 7.26 (2H, m), 7.50 (1H, d), 7.79 (1H, d), 8.34, 8.43 (4H, 2m), 9.09 (1H, t), 11.88 (1H, s); m/z (ES⁺)=358.05 [M+H]⁺; RT=3.51 min.

### In Vitro GP Activity

### Materials

α-D-Glucose-1-phosphate (disodium salt), Glycogen, D-Glucose, Malachite Green Hydrochloride, Ammonium Molybdate tetrahydrate, BSA, HEPES and rabbit muscle phosphorylase a (P1261) were purchased from Sigma. All other reagents were analytical grade.

### Method

### Glycogen Phosphorylase Assay In Vitro:

An assay for glycogen phosphorylase activity in the reverse direction was developed based on the method described by Engers et al., Can. J. Biochem., 1970, 48, 746-754]. Rabbit muscle glycogen phosphorylase a (Sigma) was reconstituted at a stock concentration of 100 μg/mL in 25 mM Tris/HCl. The pH was measured in a 96-well plate in a final volume of 100 μL containing 50 mM Hepes pH 7.2, 7.5 mM glucose, 0.5 mM glucose-1-phosphate and 1 mg/mL glycogen. After incubation at 30° C. for 30 min, the inorganic phosphate released from glucose-1-phosphate was measured by the addition of 150 μL of malachite green/molybdate solution prepared as follows: 5 mL of 4.2% ammonium molybdate in 4N HCl, 15 mL of 0.045% malachite green, 50 μL of Tween 20. Following a 30 min incubation at rt, the absorbance was measured at 620 nm. For IC₅₀ determination, 10 μL of a serial dilution of compound (100 μM to 0.004 μM) in DMSO was added to each reaction in duplicate with the equivalent concentration of DMSO added to the control uninhibited reaction. Dose response curves were then obtained by plotting % inhibition versus log₁₀ compound concentration. IC₅₀ is defined as the concentration of compound achieving 50% inhibition under the assay conditions described.

The EXAMPLES have an IC₅₀ of <1 mM. It is advantageous that the measured IC₅₀ be lower than 100 μM. It is still more advantageous for the IC₅₀ to be lower than 50 μM. It is even more advantageous for the IC₅₀ to be lower than 5 μM. It is yet more advantageous for the IC₅₀ to be lower than 0.5 μM.

What is claimed is:

1. A compound of Formula (I): 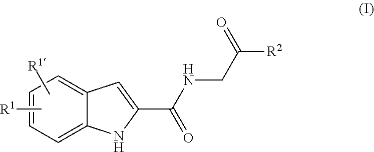
or a stereoisomer, or a pharmaceutically acceptable salt thereof, wherein:
R¹ and R^{1′} are independently selected from hydrogen, halogen, hydroxy, cyano, C₁₋₆alkyl, C₁₋₆alkoxy, fluoromethyl, difluoromethyl, trifluoromethyl, C₂₋₆alkenyl, C₂₋₆alkynyl, aryl, —C₁₋₆alkylaryl, —C₁₋₆alkylheteroaryl or aryloxy;
R² is aryl or heteroaryl, optionally substituted by up to three substituents selected from halogen, hydroxy, cyano, nitro, C₁₋₆alkyl, C₃₋₇cycloalkyl, C₂₋₆alkenyl, C₂₋₆alkynyl, C₁₋₆alkoxy, fluoromethyl, difluoromethyl, trifluoromethyl, methylenedioxo, COOR³ and NR⁴R⁵;
R³ is hydrogen or C₁₋₄alkyl; and
R⁴ and R⁵ are independently selected from hydrogen, C₁₋₄-alkyl, aryl and —C₁₋₄alkylaryl;
or R⁴ and R⁵ together with the nitrogen to which they are attached form a 4- to 7-membered heterocycle optionally containing a further heteroatom selected from N and O, which heterocycle is optionally substituted by C₁₋₄alkyl.

2. A compound according to claim 1, or a stereoisomer, or a pharmaceutically acceptable salt thereof, wherein R¹ and R^{1′} are each independently, halogen, cyano or hydrogen.

3. A compound according to claim 2, or a stereoisomer, or a pharmaceutically acceptable salt thereof, wherein one of R¹ and R^{1′} is hydrogen and the other is a 5-halo or 5-cyano group.

4. A compound according to any one of the preceding claims, or a stereoisomer, or a pharmaceutically acceptable salt thereof, wherein R² is optionally substituted phenyl, naphthyl or a 6-membered heteroaryl group any of which may be optionally substituted.

5. A compound selected from any one of Examples 1 to 10, as the free base or a pharmaceutically acceptable salt thereof.

6. A composition comprising a compound according to any one of claims 1 to 5, or a stereoisomer, or a pharmaceutically acceptable salt thereof; and a pharmaceutically acceptable carrier.

7. A method of prophylactic or therapeutic treatment of hyperglycemia or diabetes comprising a step of administering an effective amount of the compound according to any one of claims 1 to 5, or a stereoisomer, or a pharmaceutically acceptable salt thereof.

8. A method of prevention of diabetes in a human demonstrating pre-diabetic hyperglycemia or impaired glucose tolerance comprising a step of administering an effective prophylactic amount of the compound according to any one of claims 1 to 5, or a stereoisomer, or a pharmaceutically acceptable salt thereof.

9. A method of prophylactic or therapeutic treatment of hypercholesterolemia, hyperinsulinemia, hyperlipidemia, atherosclerosis or myocardial ischemia comprising a step of administering an effective amount of the compound according to any one of claims 1 to 5, or a stereoisomer, or a pharmaceutically acceptable salt thereof.

10. A method of cardioprotection comprising a step of administering to a subject in need thereof an effective amount of a compound of a compound according to any one of claims 1 to 5, or a stereoisomer or a pharmaceutically acceptable salt thereof.

11. A process for the production of a compound of Formula (I) according to claim 1, comprising:
a) removal of the ketal group of a compound of Formula (VII) in the presence of acid: 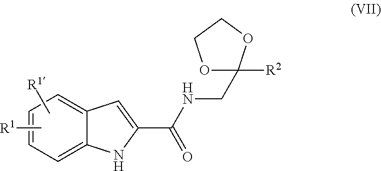
or
b) oxidation of a compound of Formula (VIII):

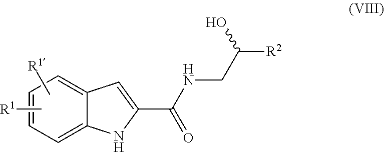

12. A compound of Formula (VII), or a protected derivative thereof: 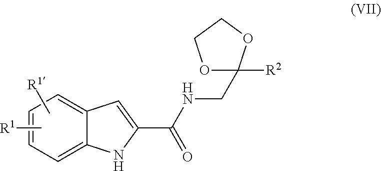
wherein R¹, R^{1′} and R² are as defined in claim 1.

13. A process for the production of a compound of Formula (VII) according to claim 12 comprising coupling of a compound of Formulae (V) with a compound of Formula (VI), or a protected or activated derivative thereof:

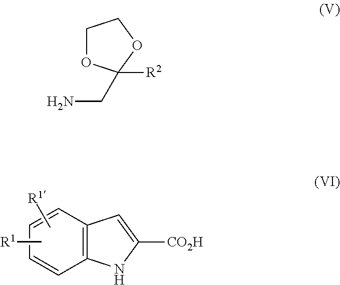

14. A compound of Formula (VIII), or a protected derivative thereof: 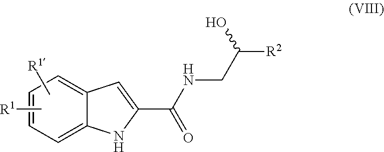
wherein R¹, R^{1′} and R² are as defined in claim 1; provided that the compound is not 5-fluoro-1H-indole-2-carboxylic acid [2-hydroxy-2-phenylethyl]amide.
